SEQUENCE LISTING (8 sequences)

SEQ ID NO: 1 - protein, 1368 aa, Streptococcus pyogenes [sequence omitted]

SEQ ID NO: 2 - protein, 1053 aa, Streptococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 3 - protein, 4 aa, Artificial Sequence
  peptide linker
GGGS

SEQ ID NO: 4 - protein, 5 aa, Artificial Sequence
  peptide linker
GGGGS

SEQ ID NO: 5 - protein, 4 aa, Artificial Sequence
  peptide linker
GGGS

SEQ ID NO: 6 - protein, 5 aa, Artificial Sequence
  peptide linker
GGGGS

SEQ ID NO: 7 - protein, 7 aa, Artificial Sequence
  SV40 large T antigen nuclear localization
PKKKRRV

SEQ ID NO: 8 - protein, 16 aa, Artificial Sequence
  nucleoplasmin nuclear localization
KRPAATKKAGQAKKKK

(19) United States
(12) Patent Application Publication
(10) Pub. No.: US 2020/0172899 A1
(43) Pub. Date: Jun. 4, 2020

(54) Epigenetically Regulated Site-Specific Nucleases

(71) Applicant: The General Hospital Corporation, Boston, MA (US)

(72) Inventors: J. Keith Joung, Winchester, MA (US); Jason Michael Gehrke, Cambridge, MA (US)

(21) Appl. No.: 16/341,563

(22) PCT Filed: Oct. 16, 2017
(86) PCT No.: PCT/US2017/056738

§ 371 Date: Apr. 12, 2019

Related U.S. Application Data
(60) Provisional application No. 62/408,645, filed on Oct. 14, 2016.

(51) Int. Cl. C12N 15/11; C12N 9/22

(52) U.S. Cl. CPC C12N 15/11; C12N 2800/80; C12N 2310/20; C12N 9/22

(57) ABSTRACT

Methods and compositions for improving the specificity of genome-editing nucleases (e.g., RNA-guided CRISPR-Cas nucleases or engineered zinc fmger nucleases) and customizable DNA-binding domain fusion proteins (e.g., RNA-guided dead-Cas9, RNA-guided dead-Cpf1, or engineered zinc finger arrays fused to transcriptional regulatory domains) for use as research reagents, in gene drives, or as therapeutic agents.

### CLAIM OF PRIORITY

This application claims the benefit of U.S. Provisional Patent Application Ser. No. 62/408,645, filed on Oct. 14, 2016. The entire contents of the foregoing are hereby incorporated by reference.

### FEDERALLY SPONSORED RESEARCH OR DEVELOPMENT

This invention was made with Government support under Grant Nos. DP1 GM105378 and R35 GM118158 awarded by the National Institutes of Health. The Government has certain rights in the invention.

### TECHNICAL FIELD

Described herein are methods and compositions for improving the specificity of genome-editing nucleases (e.g., RNA-guided CRISPR-Cas nucleases or engineered zinc finger nucleases) and customizable DNA-binding domain fusion proteins (e.g., RNA-guided dead-Cas9, RNA-guided dead-Cpf1, or engineered zinc finger arrays fused to transcriptional regulatory domains) for use as research reagents, in gene drives, or as therapeutic agents.

### BACKGROUND

Engineered targeted nucleases can be used to genetically correct disease-causing mutations in human cells. Such therapeutic strategies rely on the nuclease to introduce a sequence-specific DNA double strand break (DSB) at a specified site in the genome. For example, the specificity of RNA-guided nuclease (RGN) platforms such as CRISPR-Cas is primarily dictated by a guide RNA molecule (gRNA) bearing complementarity to the target DNA site; other genome editing platforms, like zinc-finger (ZF) nucleases or TALE nucleases, derive their specificity from sequence-specific protein-DNA contacts but require more complicated engineering strategies to produce protein domains that specifically bind to user-defined sequences. Genome editing is achieved by leveraging endogenous cell machineries that repair these targeted DSBs either via an error-prone pathway termed non-homologous end joining (NHEJ), or by more precise homology-directed repair (HDR) using a homologous exogenous “donor template” or a homologous sequence found within the genome itself. Although genome-editing nucleases can robustly induce DSBs at their specified target sites, all nuclease platforms are also known to induce unwanted DSBs at sequences that resemble the intended target. These off-target DSBs are efficiently repaired by NHEJ, resulting in unintended mutations at these sites, which can be distributed throughout the genome.

### SUMMARY

The present invention is based, at least in part, on the development of methods and compositions for improving the specificity of genome-editing nucleases (e.g., RNA-guided CRISPR-Cas nucleases or engineered zinc finger nucleases) and customizable DNA-binding domain fusion proteins (e.g., RNA-guided dead-Cas9, RNA-guided dead-Cpf1, or engineered zinc finger arrays fused to transcriptional regulatory domains) for use as research reagents, in gene drives (e.g., as described in Hammond et al., Nature Biotechnology 34:78-83 (2016)), or as therapeutic agents.

Thus, provided herein are methods for modifying the genome of a cell, comprising expressing in the cell, or contacting the cell with, a fusion protein comprising a targeted nuclease that is genetically linked to an engineered affinity protein (AP) that possesses high affinity for a specific TF or post-translational histone modification, wherein the fusion protein is only active at its target site if the specific TF or post-translational histone modification is present proximal to the target site.

In some embodiments, the AP is selected from the group consisting of single chain antibodies, engineered fibronectin domains, engineered Staphylococcus aureus immunoglobulin binding protein A, engineered nanobodies, and designed Ankyrin repeat proteins.

In some embodiments, the nuclease is selected from the group consisting of 1) meganucleases, 2) zinc-finger nucleases, 3) transcription activator effector-like nucleases (TALEN), and 4) Clustered Regularly Interspaced Short Palindromic Repeats (CRISPR)-CRISPR-associated (Cas) or CRISPR-Cpf1 RNA-guided nuclease (RGN).

In some embodiments, the nuclease is a CRISPR-Cas or CRISPR-Cpf1 RGN and the method is performed in the presence of a guide RNA.

In some embodiments, the nuclease is a Streptococcus pyogenes Cas9 nuclease harboring mutation of one or more of the residues shown in Table 1.

Also provided herein are methods for modifying the genome of a cell, comprising expressing in the cell, or contacting the cell with, a fusion protein comprising a zinc finger DNA binding domain (ZF DBD) or TAL DNA binding array fused to a Staphylococcus aureus Cas9 bearing a mutation at R1015, e.g., R1015A, R1015Q, or R1015H.

Further provided herein are methods for modifying the genome of a cell, comprising expressing in the cell, or contacting the cell with, a fusion protein comprising (i) a targeted DNA binding domain or a catalytically inactive “dead” RGN (dRGN) with a guide RNA, (ii) a heterologous functional domain, and (iii) an engineered affinity protein (AP) that is only active if the transcription factor or histone modification recognized by the AP is present proximal to the target site of the DNA binding domain or dRGN.

In some embodiments, the AP is selected from the group consisting of single chain antibodies, engineered fibronectin domains, engineered Staphylococcus aureus immunoglobulin binding protein A, engineered nanobodies, and designed Ankyrin repeat proteins.

In some embodiments, the functional domain is a transcriptional regulatory domain, a histone modifying enzyme, or a DNA modifying enzyme.

In some embodiments, the guide RNA is selected from the group consisting of (i) gRNAs with spacer lengths of 19, 18, and 17 bp; (ii) gRNAs possessing one, two, or three intentional mismatches relative to the intended target site; (iii) gRNAs with 20 nts of complementarity to the on-target site, with an additional 5′ G base (that is mismatched to the target DNA sequence) appended; and (iv) a combination of any of (i)-(iii). In some embodiments, the guide RNA is a truncated gRNA bearing very short complementarity sequences to the target DNA of 9, 10, 11, 12, or 13 nucleotide bases.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-B. RGN nuclease activity dependent on a proximal transcription factor or histone modification. (A) A representation of an affinity protein, shown here as an scFv, covalently linked to an RGN targeted to a site within a gene. Because the binding partner of the scFv isn't present at a site adjacent to the gRNA target site, the RGN is unable to induce a DSB. (B) Conversely, when the binding partner of the scFv is present adjacent to the gRNA target site, the scFv binds to its target, represented here as a transcription factor. This binding event stabilizes RGN binding at the target site, causing it to induce a DSB. This DSB can then be repaired by NHEJ or by HDR.

FIG. 2A. Characterizing the EGFP disruption activity of two SpCas9 variants with or without fusion to ZF292R, an engineered zinc finger DNA binding domain with a binding site adjacent to the gRNA target site. Both SpCas9 variants exhibit greater capacity for EGFP disruption when fused to ZF292R with all four gRNAs tested, indicating that increased binding affinity from a second DBD is sufficient to rescue activity of these SpCas9 variant-gRNA combinations.

FIG. 2B. TIDE analysis of the same cell populations from FIG. 2A confirming that both SpCas9 variants have greater capacity to cause indel formation when fused to ZF292R.

FIG. 2C. Characterizing the EGFP disruption activity of two SpCas9 variants when fused to scFv GCN4 when the proteins are expressed alone or co-expressed with GCN4-ZF292R. Both SpCas9 variants exhibit greater EGFP disruption activity when co-expressed with GCN4-ZF292R relative to when they are expressed alone with all three tested gRNAs. Activities of each of the gRNAs with wild-type SpCas9 are also shown as controls.

FIG. 3A. Characterizing the EGFP disruption activity of SpCas9 (R661A,

Q695A)-scFv GCN4 when expressed alone or co-expressed with H3 (1-38)-ZF292R or GCN4-ZF292R. Increased EGFP disruption activity by the SpCas9 variant is specific to co-expression with GCN4-ZF292R, suggesting that the interaction between GCN4-ZF292R and scFv GCN4 is mediating the increased EGFP disruption. Further, the perfectly matched gRNA5 restores SpCas9 (R661A, Q695A)-scFv GCN4 EGFP disruption activity to wild-type levels, indicating that the gRNA modifications outlined in Strategy #1 and Strategy #2 are important for inducible activity of the SpCas9 variants tested in this system.

FIG. 3B. TIDE analysis of the same cell populations from FIG. 3A demonstrating that the interaction between GCN4-ZF292R and SpCas9 (R661A, Q695A)-scFv GCN4 stimulates indel formation at the EGFP target site.

FIGS. 4A-B. (A) SpCas9 or SaCas9 variants bearing mutations that affect the protein's ability to interact with the PAM adjacent to the gRNA target site are unable to bind to, and induce DSBs at, the EGFP target site. (B) A second DBD, shown here as ZF292R, is fused to SpCas9 or SaCas9 PID KDs. The second DBD binds to a sequence adjacent to the gRNA target site, causing the Cas9 PID KD to bind its target site and induce a DSB. In this assay, when a DSB is introduced at the target site and repaired by error-prone NHEJ, the coding sequence is shifted out of frame, resulting in loss of EGFP production.

FIG. 4C. Covalently linking an engineered zinc finger DNA-binding domain to an SaCas9 PID KD can rescue its nuclease activity. Data from a representative EGFP disruption assay in which a zinc finger array binding site (ZF292R) is located 10 bp away from the PAM of an SaCas9 target site, both of which are in the coding region of EGFP. When R1015 of SaCas9 is mutated to A, Q, or H, SaCas9 proteins bearing these mutations are unable to induce DSBs. However, when ZF292R is covalently linked to the SaCas9 molecules, they are able to induce DSBs.

FIGS. 5A-B. RGN nuclease activity dependent on long-range chromatin looping. (A) A programmable DBD, represented here as a ZF array, is covalently linked to a Cas9 PID KD mutant. The DBD is targeted to a distal enhancer sequence, while the RGN is targeted to a region in the gene of interest. When the distal enhancer is not in close proximity to the gene of interest (e.g., in cell types in which the gene of interest is not transcriptionally active), the Cas9 PID KD is unable to induce a DSB at the target site. (B) However, when looping between the distal enhancer and the gene of interest occurs (e.g., in cell types in which the gene of interest is transcriptionally active), the Cas9 PID KD tethered to the enhancer via a second DBD is brought into close proximity with its target site, allowing it to induce a DSB, which is then repaired by NHEJ or HDR.

FIGS. 6A-B. (A) AP-dRGN-effector fusions (epigenome editing proteins listed in Table 1) whose DNA binding activity is dependent on interaction of the AP (here shown as a scFv protein) with a proximal transcription factor or histone modification is targeted to a genetic regulatory element (e.g., in or proximal to an enhancer, promoter, or gene body). In the absence of the AP's binding partner, the AP-dRGN-effector fusion protein is unable to stably bind to the target site specified by the gRNA and does not alter the transcriptional state of the target gene. (B) However, when the AP's binding partner, shown here as a transcription factor, is present adjacent to the gRNA target site, the binding event between the AP and its partner stabilizes the binding of the AP-dRGN-effector fusion protein. Stable recruitment of the AP-dRGN-effector protein to a target site results in modulated (e.g., activated or repressed) transcriptional output from the target gene.

### DETAILED DESCRIPTION

For therapeutic applications, a desirable capability would be to restrict nuclease activity not only to specific DNA sequences but also to only a particular epigenetic context(s), which in turn could represent a specific cell type; for example, only in cells that produce a disease phenotype or in which introduction of a genetic alteration would be expected to have a therapeutic benefit. Having such a capability would enable limitation of the number and kinds of cells in which nucleases are active, and thus minimize the number of cells in which either on- or off-target DSBs might accrue. Existing strategies for performing genome editing in a cell-type-specific manner involve ex vivo sorting approaches to separate out relevant cell types, delivering nucleic acids encoding genome editing reagents in a virus with tropism towards a specific cell or tissue type or the use of cell-type-specific regulatory elements (e.g., promoters and/or enhancers) to drive cell-type expression of the nuclease(s). Enrichment for a specific cell type by cell surface labeling and cell sorting is costly, laborious, and in some cases it may not be possible to differentiate between closely related cell types. Though some viruses have marked preference for cell type, the targetable cell types are limited and often it can be difficult to evade a neutralizing host immune response. In addition, many cell-type-specific regulatory elements such as promoters exhibit leaky expression in related cell-types, limiting their utility for genome editing applications that require tight control of nuclease activities. This strategy is also incompatible with delivery of RNA, purified nuclease proteins, or ribonucleo-protein (RNP) complexes to bulk populations of cells, strategies that have shown demonstrably lower off-target nuclease effects than delivery by DNA encoding the genome editing reagents.

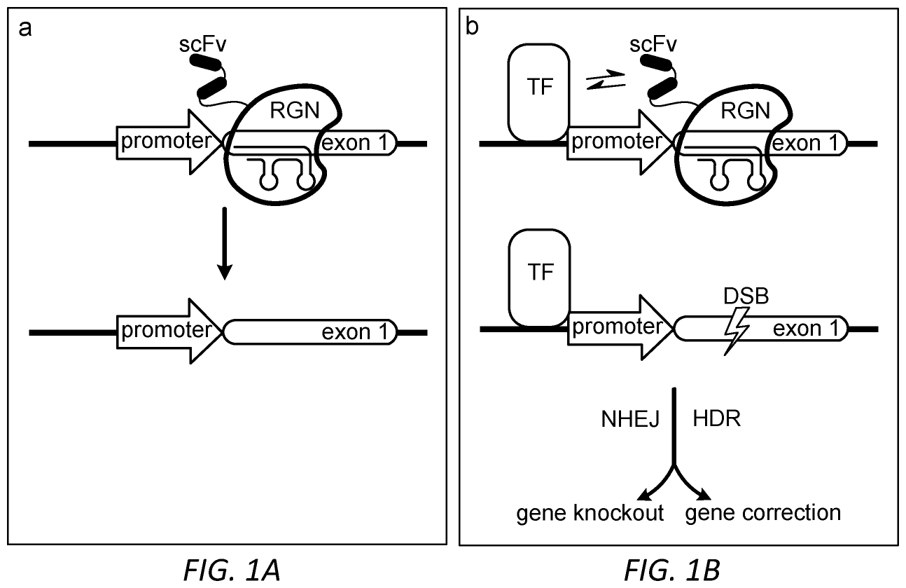

Strategy #1. Epigenetically regulated sequence-specific nucleases In one aspect, the present methods limit the activities of sequence-specific nucleases to particular cell types by engineering their cleavage activities to be dependent on the presence of specific transcription factors (TFs) or histone modifications adjacent to the target site. To do so, nucleases that on their own induce minimal or no DSBs are genetically linked to engineered affinity proteins (APs) that possess high affinities for specific TFs or post-translational histone modifications ((FIG. 1). Examples of APs include but are not limited to single chain antibodies (e.g., as described in Chothia, Cyrus, et al. “Domain association in immunoglobulin molecules: the packing of variable domains.” Journal of molecular biology 186.3 (1985): 651-663), engineered fibronectin domains (e.g., as described in Koide, Akiko, et al. “The fibronectin type III domain as a scaffold for novel binding proteins.” Journal of molecular biology 284.4 (1998): 1141-1151), engineered Staphylococcus aureus immunoglobulin binding protein A (e.g., as described in Nord, Karin, et al. “Binding proteins selected from combinatorial libraries of an a-helical bacterial receptor domain.” Nature biotechnology 15.8 (1997): 772-777), engineered nanobodies (e.g., as described in Hamers-Casterman, C. T. S. G., et al. “Naturally occurring antibodies devoid of light chains.” Nature 363.6428 (1993): 446-448), and designed Ankyrin repeat proteins (e.g., as described in Binz, H. Kaspar, et al. “Designing repeat proteins: well-expressed, soluble and stable proteins from combinatorial libraries of consensus ankyrin repeat proteins.” Journal of molecular biology 332.2 (2003): 489-503). The cleavage activities of these nuclease-AP fusions will be dependent both on recognition of the target site specified by the nuclease as well as the presence of the AP binding partner in proximity to the target site.

Specific transcription factors can include those listed herein and, for example: Hematopoietic TFs:, e.g GATA1, TAL1, ELF1, and KLF1; General transcription factors such as: factors that are members of the transcription pre-initiation complex, RNA Pol II with differential phosphorylation states of its C-terminal domain (associated with actively transcribing, paused, etc), P300 and Mediator; TFs listed under the “Affinity Protein” section below; and TFs with DNA binding motifs adjacent to regulatory elements important to specific diseases. Histone modifications include those listed here and those that are associated with different states of transcriptional activation, e.g.: H3K4me1/2/3, H3K9me1/2/3, H3K27me1/2/3, H3K9ac, H3K27ac, H3K56ac, H3K36me1/2/3, H3K79me1/2/3, or H4K16ac.

To engineer site-specific nucleases that are poised for cleavage activity (but unable to efficiently cleave their target site), binding of these nucleases to their target sites can be destabilized by (i) decreasing the non-specific affinity of the nuclease for DNA through targeted mutations to residues that contact the target DNA strands, and/or (ii) for RNA-guided nucleases such as CRISPR-Cas nucleases, engineering guide RNAs (gRNAs) with limiting or decreased affinity or interaction capability for their target sites. One specific example of such a strategy uses combinations of mutations made in the Streptococcus pyogenes Cas9 (SpCas9) nuclease that are intended to decrease affinity of the protein for DNA; examples of such mutations include but are not limited to those shown in Table 1 and any possible combinations of those mutations.

TABLE 1

| | |
|---|---|
| Cas9 (R661A, Q695A, L169A) | Cas9 (R661A, Q926A, L169A) |
| Cas9 (R661A, Q695A, Y450A) | Cas9 (R661A, Q926A, Y450A) |
| Cas9 (R661A, Q695A, M495A) | Cas9 (R661A, Q926A, M495A) |
| Cas9 (R661A, Q695A, N497A) | Cas9 (R661A, Q926A, N497A) |
| Cas9 (R661A, Q695A, M694A) | Cas9 (R661A, Q926A, M694A) |
| Cas9 (R661A, Q695A, H698A) | Cas9 (R661A, Q926A, H698A) |
| Cas9 (R661A, Q695A, K810A) | Cas9 (R661A, Q926A, K810A) |
| Cas9 (R661A, Q695A, R832A) | Cas9 (R661A, Q926A, R832A) |
| Cas9 (R661A, Q695A, D1135E) | Cas9 (R661A, Q926A, D1135E) |

Mutations in zinc fingers and ZFNs with similar effect have been described and can also be used herein; see, e.g., Guilinger et al., Nat Methods. 2014 Apr; 11(4): 429-435; Khalil et al., Cell. 2012 Aug 3;150(3):647-58.

The resulting SpCas9 variants could also be used in conjunction with gRNAs that possess decreased affinity for their genomic target sites, such as: (i) gRNAs with spacer lengths of 19, 18, and 17 bp, (ii) gRNAs possessing one, two, or three intentional mismatches relative to the intended target site, (iii) appending an additional 5′ G base (that is mismatched to the target DNA sequence) to gRNAs with 20, 19, 18, or 17 nts of complementarity to the on-target site, and (iv) a combination of any of these previously listed gRNA variations.

Strategy #2. Sequence-Specific Nucleases That Depend on Three-Dimensional Chromatin Conformation

Transcriptional regulation of many genes is controlled by the status of enhancer elements that serve to upregulate gene expression in specific contexts and cell types. These enhancers can often be very distant from the gene promoter in primary sequence, anywhere from tens to hundreds of kilobases away. However, these enhancers can be brought into close proximity with the promoter through long-range chromatin looping to activate their target genes. In this aspect, cleavage activity of nucleases is limited to specific cell types by engineering RGNs to be dependent on the occurrence of long-range chromatin looping between a regulatory element (i.e., an enhancer or the sequence surrounding an enhancer) and a target gene or gene promoter.

Previous work has shown that SpCas9 can be engineered to induce DSBs only when tethered near its target site by a second DNA binding domain (DBD) such as an engineered zinc finger array (ZF) or TALE repeat array (Bolukbasi, Mehmet Fatih, et al. “DNA-binding-domain fusions enhance the targeting range and precision of Cas9.” Nature methods 12.12 (2015): 1150-1156). This is accomplished by introducing mutations into SpCas9 at positions R1333 or R1335 that affect the ability of the protein to recognize its PAM motif (such mutants are termed Cas9 PAM interacting domain knock-downs or Cas9 PID KDs). An analogous system with SaCas9 can be engineered by fusing a second ZF DBD to a SaCas9 PID KDs bearing the mutations R1015A, R1015Q, or R1015H, which affect the interaction between SaCas9 and the PAM sequence at the target site (Kleinstiver et al., Nat Biotechnol. 2015 December; 33(12):1293-1298).

Strategy #3. Epigenetically Regulated Epigenome-Editing Proteins

Many diseases are characterized by altered expression of subsets of genes that are often causal for the disease phenotype itself. Altered gene expression is a result of specific transcription factors binding, or not binding, proximal to the promoter and/or enhancers regulating that gene in cells with the disease phenotype. Although current methods exist to modulate gene expression by genetically fusing an effector protein to programmable sequence-specific DBDs such as ZF arrays, TALE repeat arrays, and catalytically inactive RGNs (dead RGNs or dRGNs), these tools are expected to function in all cell types to which the reagents are delivered and do not have intrinsic specificity for cells with specific disease or non-disease phenotypes. As a result, delivering these reagents to desired subsets of cells requires complicated ex vivo approaches or expressing these reagents from cell-type-specific transcriptional regulatory elements, a strategy incompatible with protein delivery. In this aspect, gene expression is modified in a manner conditional on the presence of specific TFs or histone modifications located proximal to the gene of interest, resulting in the programmed modulation of a gene's expression only in cells with a specific TF binding or histone modification profile.

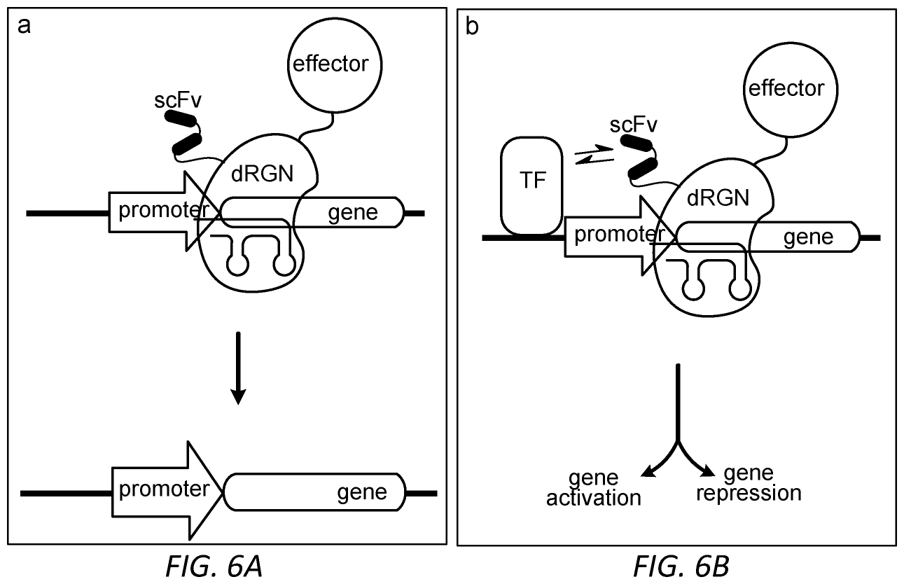

For example, the methods can include using dRGNs, with or without modifications intended to reduce non-specific affinity for DNA listed in Strategies #1 and #2, genetically fused to APs and to effector proteins (heterologous functional domains) that are able to alter the transcriptional output of genes (Table 2). These dRGNs will be used with various modified gRNAs (e.g., those outlined in Strategies #1 and #2) that in complex with the dRGN are unable to stably bind to the target site specified by the gRNA sequence. However, when the binding partner to the AP (e.g. the specified TF or histone modification) is also present in close proximity to the gRNA binding site, the increased affinity for the target site from the AP-binding partner interaction allows the complex to stably associate with the specified target site (FIGS. 6A and 6B). The effector fused to the dRGN-AP is then able to alter the expression of the target gene. In addition to the modified gRNAs listed in Strategies #1 and #2, we also propose using dRGN proteins bearing only catalytically-inactivating mutations (i.e. without additional mutations intended to decrease non-specific affinity for DNA) with gRNAs bearing very short spacer sequences of 9, 10, 11, 12, or 13 nucleotide bases. Because this strategy requires only stable binding of the dRGN complex to a target site and not nuclease activity, gRNAs bearing 9-13 base spacer sequences are likely to be sufficient to enable the complex to bind in conjunction with the AP-binding partner interaction.

TABLE 2

| Effector Protein | Effect on Gene Expression |
|---|---|
| SID domain | Repression |
| KRAB domain | Repression |
| DNMT3A (full length protein or catalytic | Repression |
| domain) | |
| LSD1 (full length protein or catalytic | Repression |
| domain) | |
| VP16 or VP64 | Activation |
| P300 (full length protein or catalytic | Activation |
| domain) | |
| TET1 (full length protein or catalytic | Activation |
| domain) | |

Engineered Affinity Proteins (APs)

APs useful in the present fusion proteins are those that possess high affinity for a specific transcription factor (TF) or post-translational histone modifications (e.g., as shown in FIG. 1). Examples of APs include but are not limited to single chain antibodies, engineered fibronectin domains, engineered Staphylococcus aureus immunoglobulin binding protein A, engineered nanobodies, and designed Ankyrin repeat proteins. Examples of TFs include the general transcription factors (e.g., TFIIA, TFIIB, TFIID, TFIIE, TFIIF, and TFIIH); developmentally regulated TFs (e.g., GATA, HNF, PIT-1, MyoD, Myf5, Hox, Winged Helix); and signal-dependent TFs (e.g., SP1, AP-1, C/EBP, heat shock factor, ATF/CREB, c-Myc, MEF2, STAT, R-SMAD, NF-κB, Notch, TUBBY, NFAT, and SREBP). Examples of specific post-translational histone modifications include methylation, phosphorylation, acetylation, ubiquitylation, and sumoylation. These can be targeted via engineered proteins with specific affinity to these modifications made to these proteins.

Specific transcription factors can include those listed above and, for example: Hematopoietic TFs:, e.g GATA1, TALI, ELF1, and KLF1; General transcription factors such as: factors that are members of the transcription pre-initiation complex, RNA Pol II with differential phosphorylation states of its C-terminal domain (associated with actively transcribing, paused, etc), P300 and Mediator; TFs listed under the “Affinity Protein” section below; and TFs with DNA binding motifs adjacent to regulatory elements important to specific diseases. Histone modifications include those listed here and those that are associated with different states of transcriptional activation, e.g.: H3K4me1/2/3, H3K9me1/2/3, H3K27me1/2/3, H3K9ac, H3K27ac, H3K56ac, H3K36me1/2/3, H3K79me1/2/3, or H4K16ac.

Sequence-Specific Nucleases

There are presently four main classes of sequence-specific nucleases: 1) meganucleases, 2) zinc-finger nucleases, 3) transcription activator effector-like nucleases (TALEN), and 4) Clustered Regularly Interspaced Short Palindromic Repeats (CRISPR) Cas RNA-guided nucleases (RGN). Modifications of these proteins can be made to knock down non-specific affinity of the protein for DNA such that the protein is unable to stably bind its target sequence without additional binding energy from the affinity protein-binding partner. For ZFNs, residues in the ZF domains that contact the phosphate DNA backbone could be knocked out (see Khalil et al., Cell 2012). For TALEs, there is a specific residue in each repeat that mediates DNA phosphate contacts that could be mutated. In some embodiments, 3-finger ZF arrays with a knocked down nuclease domain or short TALEN arrays (e.g. 7.5 or 8.5) for less binding energy such that only very long binding events leads to nuclease activity can be used. Various components of these platforms can also be fused together to create additional nucleases such as Mega-TALs and FokI-dCas9 fusions. See, e.g., Gaj et al., Trends Biotechnol. 2013 July; 31(7):397-405. The nuclease can be transiently or stably expressed in the cell, using methods known in the art; typically, to obtain expression, a sequence encoding a protein is subcloned into an expression vector that contains a promoter to direct transcription. Suitable eukaryotic expression systems are well known in the art and described, e.g., in Sambrook et al., Molecular Cloning, A Laboratory Manual (4th ed. 2013); Kriegler, Gene Transfer and Expression: A Laboratory Manual (2006); and Current Protocols in Molecular Biology (Ausubel et al., eds., 2010). Transformation of eukaryotic and prokaryotic cells are performed according to standard techniques (see, e.g., the reference above and Morrison, 1977, J. Bacteriol. 132:349-351; Clark-Curtiss & Curtiss, Methods in Enzymology 101:347-362 (Wu et al., eds, 1983).

Homing Meganucleases

Meganucleases are sequence-specific endonucleases originating from a variety of organisms such as bacteria, yeast, algae and plant organelles. Endogenous meganucleases have recognition sites of 12 to 30 base pairs; customized DNA binding sites with 18 bp and 24 bp-long meganuclease recognition sites have been described, and either can be used in the present methods and constructs. See, e.g., Silva, G, et al., Current Gene Therapy, 11:11-27, (2011); Arnould et al., Journal of Molecular Biology, 355:443-58 (2006); Arnould et al., Protein Engineering Design & Selection, 24:27-31 (2011); and Stoddard, Q. Rev. Biophys. 38, 49 (2005); Grizot et al., Nucleic Acids Research, 38:2006-18 (2010).

CRISPR-Cas Nucleases

Recent work has demonstrated that clustered, regularly interspaced, short palindromic repeats (CRISPR)/CRISPR-associated (Cas) systems (Wiedenheft et al., Nature 482, 331-338 (2012); Horvath et al., Science 327, 167-170 (2010); Terns et al., Curr Opin Microbiol 14, 321-327 (2011)) can serve as the basis of a simple and highly efficient method for performing genome editing in bacteria, yeast and human cells, as well as in vivo in whole organisms such as fruit flies, zebrafish and mice (Wang et al., Cell 153, 910-918 (2013); Shen et al., Cell Res (2013); Dicarlo et al., Nucleic Acids Res (2013); Jiang et al., Nat Biotechnol 31, 233-239 (2013); Jinek et al., Elife 2, e00471 (2013); Hwang et al., Nat Biotechnol 31, 227-229 (2013); Cong et al., Science 339, 819-823 (2013); Mali et al., Science 339, 823-826 (2013c); Cho et al., Nat Biotechnol 31, 230-232 (2013); Gratz et al., Genetics 194(4):1029-35 (2013)). The Cas9 nuclease from S. pyogenes (hereafter simply Cas9) can be guided via simple base pair complementarity between 17-20 nucleotides of an engineered guide RNA (gRNA), e.g., a single guide RNA or crRNA/tracrRNA pair, and the complementary strand of a target genomic DNA sequence of interest that lies next to a protospacer adjacent motif (PAM), e.g., a PAM matching the sequence NGG or NAG (Shen et al., Cell Res (2013); Dicarlo et al., Nucleic Acids Res (2013); Jiang et al., Nat Biotechnol 31, 233-239 (2013); Jinek et al., Elife 2, e00471 (2013); Hwang et al., Nat Biotechnol 31, 227-229 (2013); Cong et al., Science 339, 819-823 (2013); Mali et al., Science 339, 823-826 (2013c); Cho et al., Nat Biotechnol 31, 230-232 (2013); Jinek et al., Science 337, 816-821 (2012)). The engineered CRISPR from Prevotella and Francisella 1 (Cpf1) nuclease can also be used, e.g., as described in Zetsche et al., Cell 163, 759-771 (2015); Schunder et al., Int J Med Microbiol 303, 51-60 (2013);

Makarova et al., Nat Rev Microbiol 13, 722-736 (2015); Fagerlund et al., Genome Biol 16, 251 (2015). Unlike SpCas9, Cpf1 requires only a single 42-nt crRNA, which has 23 nt at its 3′ end that are complementary to the protospacer of the target DNA sequence (Zetsche et al., 2015). Furthermore, whereas SpCas9 recognizes an NGG PAM sequence that is 3′ of the protospacer, AsCpf1 and LbCp1 recognize TTTN PAMs that are found 5′ of the protospacer (Id.).

In some embodiments, the present system utilizes a wild type or variant Cas9 protein from S. pyogenes or Staphylococcus aureus, or a wild type Cpf1 protein from Acidaminococcus sp. BV3L6 or Lachnospiraceae bacterium ND2006 either as encoded in bacteria or codon-optimized for expression in mammalian cells and/or modified in its PAM recognition specificity and/or its genome-wide specificity. A number of variants have been described; see, e.g., WO 2016/141224, PCT/US2016/049147, Kleinstiver et al., Nat Biotechnol. 2016 August; 34(8):869-74; Tsai and Joung, Nat Rev Genet. 2016 May; 17(5):300-12; Kleinstiver et al., Nature. 2016 Jan. 28; 529(7587):490-5; Shmakov et al., Mol Cell. 2015 Nov. 5; 60(3):385-97; Kleinstiver et al., Nat Biotechnol. 2015 December; 33(12):1293-1298; Dahlman et al., Nat Biotechnol. 2015 November; 33(11):1159-61; Kleinstiver et al., Nature. 2015 July 23; 523(7561):481-5; Wyvekens et al., Hum Gene Ther. 2015 July; 26(7):425-31; Hwang et al., Methods Mol Biol. 2015; 1311:317-34; Osborn et al., Hum Gene Ther. 2015 February; 26(2):114-26; Konermann et al., Nature. 2015 Jan. 29; 517(7536):583-8; Fu et al., Methods Enzymol. 2014; 546:21-45; and Tsai et al., Nat Biotechnol. 2014 June; 32(6):569-76, inter alia. The guide RNA is expressed or present in the cell together with the Cas9 or Cpf1. Either the guide RNA or the nuclease, or both, can be expressed transiently or stably in the cell or introduced as a purified protein or nucleic acid.

In some embodiments, the SpCas9 also include one of the following mutations, which reduce or destroy the nuclease activity of the Cas9: D10, E762, D839, H983, or D986 and H840 or N863, e.g., D10A/D10N and H840A/H840N/H840Y, to render the nuclease portion of the protein catalytically inactive; substitutions at these positions could be alanine (as they are in Nishimasu al., Cell 156, 935-949 (2014)), or other residues, e.g., glutamine, asparagine, tyrosine, serine, or aspartate, e.g., E762Q, H983N, H983Y, D986N, N863D, N863S, or N863H (see WO 2014/152432). In some embodiments, the variant includes mutations at D10A or H840A (which creates a single-strand nickase), or mutations at D10A and H840A (which abrogates nuclease activity; this mutant is known as dead Cas9 or dCas9).

In some embodiments, the nuclease is a FokI-dCas9 fusion, RNA-guided FokI nucleases in which Cas9 nuclease has been rendered catalytically inactive by mutation (e.g., dCas9) and a FokI nuclease fused in frame, optionally with an intervening linker, to the dCas9. See, e.g., WO 2014/144288 and WO 2014/204578.

The methods can include the use of a wild-type Cas protein with normal affinity for the DNA with a guide RNA that has reduced affinity, e.g., (1) gRNA with 20 nt of homology to the target site and with an additional 5′ appended G that is mismatched to the target site sequence; (2) gRNA with 19 nt of homology to the target site and a 5′ 20th nt that is a G, which is mismatched to the target site; or (3) gRNA with 18 nt of homology to the target site with two 5′ Gs mismatched to the target site. Known methods can be modified for designing and making suitable guide RNAs, e.g., as described in any of the references above.

Thus, provided herein are Cas9 variants, including SpCas9 variants. The SpCas9 wild type sequence is as follows:

```
(SEQ ID NO: 1)
                10 20 30 40 50 60
MDKKYSIGLD IGTNSVGWAV ITDEYKVPSK KFKVLGNTDR HSIKKNLIGA LLFDSGETAE
                70 80 90 100 110 120
ATRLKRTARR RYTRRKNRIC YLQEIFSNEM AKVDDSFFHR LEESFLVEED KKHERHPIFG
              130 140 150 160 170 180
NIVDEVAYHE KYPTIYHLRK KLVDSTDKAD LRLIYLALAH MIKFRGHFLI EGDLNPDNSD
              190 200 210 220 230 240
VDKLFIQLVQ TYNQLFEENP INASGVDAKA ILSARLSKSR RLENLIAQLP GEKKNGLFGN
              250 260 270 280 290 300
LIALSLGLTP NFKSNFDLAE DAKLQLSKDT YDDDLDNLLA QIGDQYADLF LAAKNLSDAI
              310 320 330 340 350 360
LLSDILRVNT EITKAPLSAS MIKRYDEHHQ DLTLLKALVR QQLPEKYKEI FFDQSKNGYA
              370 380 390 400 410 420
GYIDGGASQE EFYKFIKPIL EKMDGTEELL VKLNREDLLR KQRTFDNGSI PHQIHLGELH
              430 440 450 460 470 480
AILRRQEDFY PFLKDNREKI EKILTFRIPY YVGPLARGNS RFAWMTRKSE ETITPWNFEE
              490 500 510 520 530 540
VVDKGASAQS FIERMTNFDK NLPNEKVLPK HSLLYEYFTV YNELTKVKYV TEGMRKPAFL
              550 560 570 580 590 600
SGEQKKAIVD LLFKTNRKVT VKQLKEDYFK KIECFDSVEI SGVEDRFNAS LGTYHDLLKI
              610 620 630 640 650 660
IKDKDFLDNE ENEDILEDIV LTLTLFEDRE MIEERLKTYA HLFDDKVMKQ LKRRRYTGWG
              670 680 690 700 710 720
RLSRKLINGI RDKQSGKTIL DFLKSDGFAN RNFMQLIHDD SLTFKEDIQK AQVSGQGDSL
              730 740 750 760 770 780
HEHIANLAGS PAIKKGILQT VKVVDELVKV MGRHKPENIV IEMARENQTT QKGQKNSRER
              790 800 810 820 830 840
MKRIEEGIKE LGSQILKEHP VENTQLQNEK LYLYYLQNGR DMYVDQELDI NRLSDYDVDH
              850 860 870 880 890 900
IVPQSFLKDD SIDNKVLTRS DKNRGKSDNV PSEEVVKKMK NYWRQLLNAK LITQRKFDNL
              910 920 930 940 950 960
TKAERGGLSE LDKAGFIKRQ LVETRQITKH VAQILDSRMN TKYDENDKLI REVKVITLKS
              970 980 990 1000 1010 1020
KLVSDFRKDF QFYKVREINN YHHAHDAYLN AVVGTALIKK YPKLESEFVY GDYKVYDVRK
            1030 1040 1050 1060 1070 1080
MIAKSEQEIG KATAKYFFYS NIMNFFKTEI TLANGEIRKR PLIETNGETG EIVWDKGRDF
            1090 1100 1110 1120 1130 1140
ATVRKVLSMP QVNIVKKTEV QTGGFSKESI LPKRNSDKLI ARKKDWDPKK YGGFDSPTVA
            1150 1160 1170 1180 1190 1200
YSVLVVAKVE KGKSKKLKSV KELLGITIME RSSFEKNPID FLEAKGYKEV KKDLIIKLPK
            1210 1220 1230 1240 1250 1260
YSLFELENGR KRMLASAGEL QKGNELALPS KYVNFLYLAS HYEKLKGSPE DNEQKQLFVE
            1270 1280 1290 1300 1310 1320
QHKHYLDEII EQISEFSKRV ILADANLDKV LSAYNKHRDK PIREQAENII HLFTLTNLGA
            1330 1340 1350 1360
PAAFKYFDTT IDRKRYTSTK EVLDATLIHQ SITGLYETRI DLSQLGGD
```

The SpCas9 variants described herein can include the amino acid sequence of SEQ ID NO:1, with mutations (i.e., replacement of the native amino acid with a different amino acid, e.g., alanine, glycine, or serine), as described herein or known in the art. In some embodiments, the SpCas9 variants are at least 80%, e.g., at least 85%, 90%, or 95% identical to the amino acid sequence of SEQ ID NO:1, e.g., have differences at up to 5%, 10%, 15%, or 20% of the residues of SEQ ID NO:1 replaced, e.g., with conservative mutations, in addition to the mutations described herein.

Also provided herein are SaCas9 variants. The SaCas9 wild type sequence is as follows:

```
(SEQ ID NO: 2)
                10 20 30 40 50
MKRNYILGLD IGITSVGYGI IDYETRDVID AGVRLFKEAN VENNEGRRSK
                60 70 80 90 100
RGARRLKRRR RHRIQRVKKL LFDYNLLTDH SELSGINPYE ARVKGLSQKL
              110 120 130 140 150
SEEEFSAALL HLAKRRGVHN VNEVEEDTGN ELSTKEQISR NSKALEEKYV
              160 170 180 190 200
AELQLERLKK DGEVRGSINR FKTSDYVKEA KQLLKVQKAY HQLDQSFIDT
              210 220 230 240 250
YIDLLETRRT YYEGPGEGSP FGWKDIKEWY EMLMGHCTYF PEELRSVKYA
              260 270 280 290 300
YNADLYNALN DLNNLVITRD ENEKLEYYEK FQIIENVFKQ KKKPTLKQIA
              310 320 330 340 350
KEILVNEEDI KGYRVTSTGK PEFTNLKVYH DIKDITARKE IIENAELLDQ
              360 370 380 390 400
IAKILTIYQS SEDIQEELTN LNSELTQEEI EQISNLKGYT GTHNLSLKAI
              410 420 430 440 450
NLILDELWHT NDNQIAIFNR LKLVPKKVDL SQQKEIPTTL VDDFILSPVV
              460 470 480 490 500
KRSFIQSIKV INAIIKKYGL PNDIIIELAR EKNSKDAQKM INEMQKRNRQ
              510 520 530 540 550
TNERIEEIIR TTGKENAKYL IEKIKLHDMQ EGKCLYSLEA IPLEDLLNNP
              560 570 580 590 600
FNYEVDHIIP RSVSFDNSFN NKVLVKQEEN SKKGNRTPFQ YLSSSDSKIS
              610 620 630 640 650
YETFKKHILN LAKGKGRISK TKKEYLLEER DINRFSVQKD FINRNLVDTR
              660 670 680 690 700
YATRGLMNLL RSYFRVNNLD VKVKSINGGF TSFLRRKWKF KKERNKGYKH
              710 720 730 740 750
HAEDALIIAN ADFIFKEWKK LDKAKKVMEN QMFEEKQAES MPEIETEQEY
              760 770 780 790 800
KEIFITPHQI KHIKDFKDYK YSHRVDKKPN RELINDTLYS TRKDDKGNTL
              810 820 830 840 850
IVNNLNGLYD KDNDKLKKLI NKSPEKLLMY HHDPQTYQKL KLIMEQYGDE
              860 870 880 890 900
KNPLYKYYEE TGNYLTKYSK KDNGPVIKKI KYYGNKLNAH LDITDDYPNS
              910 920 930 940 950
RNKVVKLSLK PYRFDVYLDN GVYKFVTVKN LDVIKKENYY EVNSKCYEEA
              960 970 980 990 1000
KKLKKISNQA EFIASFYNND LIKINGELYR VIGVNNDLLN RIEVNMIDIT
            1010 1020 1030 1040 1050
YREYLENMND KRPPRIIKTI ASKTQSIKKY STDILGNLYE VKSKKHPQII
KKG
```

SaCas9 variants described herein include the amino acid sequence of SEQ ID NO:2, with mutations as described herein or known in the art, e.g., comprising a sequence that is at least 80%, e.g., at least 85%, 90%, or 95%, identical to the amino acid sequence of SEQ ID NO:2 with mutations described herein or known in the art.

To determine the percent identity of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences. Percent identity between two polypeptides or nucleic acid sequences is determined in various ways that are within the skill in the art, for instance, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed, pp 353-358; BLAST program (Basic Local Alignment Search Tool; (Altschul, S. F., W. Gish, et al. (1990) J Mol Biol 215: 403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for proteins or nucleic acids, the length of comparison can be any length, up to and including full length (e.g., 5%, 10%, 20%, 30%, 40%, 50%, 60%, 70%, 80%, 90%, 95%, or 100%). For purposes of the present compositions and methods, at least 80% of the full length of the sequence is aligned.

For purposes of the present invention, the comparison of sequences and determination of percent identity between two sequences can be accomplished using a Blossum 62 scoring matrix with a gap penalty of 12, a gap extend penalty of 4, and a frameshift gap penalty of 5.

Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

TAL Effector Repeat Arrays

TAL effectors of plant pathogenic bacteria in the genus Xanthomonas play important roles in disease, or trigger defense, by binding host DNA and activating effector-specific host genes. Specificity depends on an effector-variable number of imperfect, typically ˜33-35 amino acid repeats. Polymorphisms are present primarily at repeat positions 12 and 13, which are referred to herein as the repeat variable-diresidue (RVD). The RVDs of TAL effectors correspond to the nucleotides in their target sites in a direct, linear fashion, one RVD to one nucleotide, with some degeneracy and no apparent context dependence. In some embodiments, the polymorphic region that grants nucleotide specificity may be expressed as a triresidue or triplet.

Each DNA binding repeat can include a RVD that determines recognition of a base pair in the target DNA sequence, wherein each DNA binding repeat is responsible for recognizing one base pair in the target DNA sequence. In some embodiments, the RVD can comprise one or more of: HA for recognizing C; ND for recognizing C; HI for recognizing C; HN for recognizing G; NA for recognizing G; SN for recognizing G or A; YG for recognizing T; and NK for recognizing G, and one or more of: HD for recognizing C; NG for recognizing T; NI for recognizing A; NN for recognizing G or A; NS for recognizing A or C or G or T; N* for recognizing C or T, wherein * represents a gap in the second position of the RVD; HG for recognizing T; H* for recognizing T, wherein * represents a gap in the second position of the RVD; and IG for recognizing T.

TALE proteins may be useful in research and biotechnology as targeted chimeric nucleases that can facilitate homologous recombination in genome engineering (e.g., to add or enhance traits useful for biofuels or biorenewables in plants). These proteins also may be useful as, for example, transcription factors, and especially for therapeutic applications requiring a very high level of specificity such as therapeutics against pathogens (e.g., viruses) as non-limiting examples.

Methods for generating engineered TALE arrays are known in the art, see, e.g., the fast ligation-based automatable solid-phase high-throughput (FLASH) system described in U.S. Ser. No. 61/610,212, and Reyon et al., Nature Biotechnology 30,460-465 (2012); as well as the methods described in Bogdanove & Voytas, Science 333, 1843-1846 (2011); Bogdanove et al., Curr Opin Plant Biol 13, 394-401 (2010); Scholze & Boch, J. Curr Opin Microbiol (2011); Boch et al., Science 326, 1509-1512 (2009); Moscou & Bogdanove, Science 326, 1501 (2009); Miller et al., Nat Biotechnol 29, 143-148 (2011); Morbitzer et al., T. Proc Natl Acad Sci USA 107, 21617-21622 (2010); Morbitzer et al., Nucleic Acids Res 39, 5790-5799 (2011); Zhang et al., Nat Biotechnol 29, 149-153 (2011); Geissler et al., PLoS ONE 6, e19509 (2011); Weber et al., PLoS ONE 6, e19722 (2011); Christian et al., Genetics 186, 757-761 (2010); Li et al., Nucleic Acids Res 39, 359-372 (2011); Mahfouz et al., Proc Natl Acad Sci USA 108, 2623-2628 (2011); Mussolino et al., Nucleic Acids Res (2011); Li et al., Nucleic Acids Res 39, 6315-6325 (2011); Cermak et al., Nucleic Acids Res 39, e82 (2011); Wood et al., Science 333, 307 (2011); Hockemeye et al. Nat Biotechnol 29, 731-734 (2011); Tesson et al., Nat Biotechnol 29, 695-696 (2011); Sander et al., Nat Biotechnol 29, 697-698 (2011); Huang et al., Nat Biotechnol 29, 699-700 (2011); and Zhang et al., Nat Biotechnol 29, 149-153 (2011); all of which are incorporated herein by reference in their entirety.

Also suitable for use in the present methods are MegaTALs, which are a fusion of a meganuclease with a TAL effector; see, e.g., Boissel et al., Nucl. Acids Res. 42(4):2591-2601 (2014); Boissel and Scharenberg, Methods Mol Biol. 2015; 1239:171-96.

The TALs can be fused to functional domains, such as transcriptional activators, transcriptional repressors, methylation domains (e.g., a catalytic domain comprising a sequence that catalyzes hydroxylation of methylated cytosines in DNA, see WO2013181228), and nucleases to regulate gene expression, alter DNA methylation, and to introduce targeted alterations into genomes of model organisms, plants, and human cells. See, e.g., Tan et al., PNAS 100:11997-12002 (2003); Wong et al., Cancer Res. 59:71-73 (1999); Zhang et al., Nat. Biotech. 29:149-154 (2011); and WO2013181228.

Zinc Fingers

Zinc finger proteins are DNA-binding proteins that contain one or more zinc fingers, independently folded zinc-containing mini-domains, the structure of which is well known in the art and defined in, for example, Miller et al., 1985, EMBO J., 4:1609; Berg, 1988, Proc. Natl. Acad. Sci. USA, 85:99; Lee et al., 1989, Science. 245:635; and Klug, 1993, Gene, 135:83. Crystal structures of the zinc finger protein Zif268 and its variants bound to DNA show a semi-conserved pattern of interactions, in which typically three amino acids from the alpha-helix of the zinc finger contact three adjacent base pairs or a “subsite” in the DNA (Pavletich et al., 1991, Science, 252:809; Elrod-Erickson et al., 1998, Structure, 6:451). Thus, the crystal structure of Zif268 suggested that zinc finger DNA-binding domains might function in a modular manner with a one-to-one interaction between a zinc finger and a three-base-pair “subsite” in the DNA sequence. In naturally occurring zinc finger transcription factors, multiple zinc fingers are typically linked together in a tandem array to achieve sequence-specific recognition of a contiguous DNA sequence (Klug, 1993, Gene 135:83).

Multiple studies have shown that it is possible to artificially engineer the DNA binding characteristics of individual zinc fingers by randomizing the amino acids at the alpha-helical positions involved in DNA binding and using selection methodologies such as phage display to identify desired variants capable of binding to DNA target sites of interest (Rebar et al., 1994, Science, 263:671; Choo et al., 1994 Proc. Natl. Acad. Sci. USA, 91:11163; Jamieson et al., 1994, Biochemistry 33:5689; Wu et al., 1995 Proc. Natl. Acad. Sci. USA, 92: 344). Such recombinant zinc finger proteins can be fused to functional domains, such as transcriptional activators, transcriptional repressors, methylation domains, and nucleases to regulate gene expression, alter DNA methylation, and introduce targeted alterations into genomes of model organisms, plants, and human cells (Carroll, 2008, Gene Ther., 15:1463-68; Cathomen, 2008, Mol. Ther., 16:1200-07; Wu et al., 2007, Cell. Mol. Life Sci., 64:2933-44).

One existing method for engineering zinc finger arrays, known as “modular assembly,” advocates the simple joining together of pre-selected zinc finger modules into arrays (Segal et al., 2003, Biochemistry, 42:2137-48; Beerli et al., 2002, Nat. Biotechnol., 20:135-141; Mandell et al., 2006, Nucleic Acids Res., 34:W516-523; Carroll et al., 2006, Nat. Protoc. 1:1329-41; Liu et al., 2002, J. Biol. Chem., 277:3850-56; Bae et al., 2003, Nat. Biotechnol., 21:275-280; Wright et al., 2006, Nat. Protoc., 1:1637-52). Although straightforward enough to be practiced by any researcher, recent reports have demonstrated a high failure rate for this method, particularly in the context of zinc finger nucleases (Ramirez et al., 2008, Nat. Methods, 5:374-375; Kim et al., 2009, Genome Res. 19:1279-88), a limitation that typically necessitates the construction and cell-based testing of very large numbers of zinc finger proteins for any given target gene (Kim et al., 2009, Genome Res. 19:1279-88).

Combinatorial selection-based methods that identify zinc finger arrays from randomized libraries have been shown to have higher success rates than modular assembly (Maeder et al., 2008, Mol. Cell, 31:294-301; Joung et al., 2010, Nat. Methods, 7:91-92; Isalan et al., 2001, Nat. Biotechnol., 19:656-660). In preferred embodiments, the zinc finger arrays are described in, or are generated as described in, WO 2011/017293 and WO 2004/099366. Additional suitable zinc finger DBDs are described in U.S. Pat. Nos. 6,511,808, 6,013,453, 6,007,988, and 6,503,717 and U.S. patent application 2002/0160940.

Heterologous Functional Domains

In some embodiments, the fusion proteins described herein includes a heterologous functional domain as described in U.S. Pat. No. 8,993,233; US 20140186958; U.S. Pat. No. 9,023,649; WO/2014/099744; WO 2014/089290; WO2014/144592; WO144288; WO2014/204578; WO2014/152432; WO2115/099850; U.S. Pat. No. 8,697,359; US2010/0076057; US2011/0189776; US2011/0223638; US2013/0130248; WO/2008/108989; WO/2010/054108; WO/2012/164565; WO/2013/098244; WO/2013/176772; US20150050699; US 20150071899 and WO 2014/124284. IN preferred embodiments, the heterologous functional domain alters DNA. For example, the nuclease, preferably comprising one or more nuclease activity-reducing or killing mutation, and/or one or more mutation that reduces DNA binding affinity, can be fused to a transcriptional activation domain or other heterologous functional domains (e.g., transcriptional repressors (e.g., KRAB, ERD, SID, and others, e.g., amino acids 473-530 of the ets2 repressor factor (ERF) repressor domain (ERD), amino acids 1-97 of the KRAB domain of KOX1, or amino acids 1-36 of the Mad mSIN3 interaction domain (SID); see Beerli et al., PNAS USA 95:14628-14633 (1998)) or silencers such as Heterochromatin Protein 1 (HP1, also known as swi6), e.g., HP1α or HP1β; proteins or peptides that could recruit long non-coding RNAs (lncRNAs) fused to a fixed RNA binding sequence such as those bound by the MS2 coat protein, endoribonuclease Csy4, or the lambda N protein; enzymes that modify the methylation state of DNA (e.g., DNA methyltransferase (DNMT) or TET proteins); or enzymes that modify histone subunits (e.g., histone acetyltransferases (HAT), histone deacetylases (HDAC), histone methyltransferases (e.g., for methylation of lysine or arginine residues) or histone demethylases (e.g., for demethylation of lysine or arginine residues)) as are known in the art can also be used. A number of sequences for such domains are known in the art, e.g., a domain that catalyzes hydroxylation of methylated cytosines in DNA. Exemplary proteins include the Ten-Eleven-Translocation (TET)1-3 family, enzymes that converts 5-methylcytosine (5-mC) to 5-hydroxymethylcytosine (5-hmC) in DNA.

Sequences for human TET1-3 are known in the art and are shown in the following table:

GenBank Accession Nos.

| Gene | Amino Acid | Nucleic Acid |
|---|---|---|
| TET1 | NP_085128.2 | NM_030625.2 |
| TET2* | NP_001120680.1 (var 1) | NM_001127208.2 |
| | NP_060098.3 (var 2) | NM_017628.4 |
| TET3 | NP_659430.1 | NM_144993.1 |

*Variant (1) represents the longer transcript and encodes the longer isoform (a). Variant (2) differs in the 5' UTR and in the 3′ UTR and coding sequence compared to variant 1. The resulting isoform (b) is shorter and has a distinct C-terminus compared to isoform a.

In some embodiments, all or part of the full-length sequence of the catalytic domain can be included, e.g., a catalytic module comprising the cysteine-rich extension and the 2OGFeDO domain encoded by 7 highly conserved exons, e.g., the Tet1 catalytic domain comprising amino acids 1580-2052, Tet2 comprising amino acids 1290-1905 and Tet3 comprising amino acids 966-1678. See, e.g., FIG. 1 of Iyer et al., Cell Cycle. 2009 Jun. 1; 8(11):1698-710. Epub 2009 Jun. 27, for an alignment illustrating the key catalytic residues in all three Tet proteins, and the supplementary materials thereof (available at ftp site ftp.ncbi.nih.gov/pub/aravind/DONS/supplementary_material_DONS.html) for full length sequences (see, e.g., seq 2c); in some embodiments, the sequence includes amino acids 1418-2136 of Tet1 or the corresponding region in Tet2/3.

Other catalytic modules can be from the proteins identified in Iyer et al., 2009.

In some embodiments, the heterologous functional domain is a biological tether, and comprises all or part of (e.g., DNA binding domain from) the MS2 coat protein, endoribonuclease Csy4, or the lambda N protein. These proteins can be used to recruit RNA molecules containing a specific stem-loop structure to a locale specified by the dCas9 gRNA targeting sequences. For example, a dCas9 variant fused to MS2 coat protein, endoribonuclease Csy4, or lambda N can be used to recruit a long non-coding RNA (IncRNA) such as XIST or HOTAIR; see, e.g., Keryer-Bibens et al., Biol. Cell 100:125-138 (2008), that is linked to the Csy4, MS2 or lambda N binding sequence. Alternatively, the Csy4, MS2 or lambda N protein binding sequence can be linked to another protein, e.g., as described in Keryer-Bibens et al., supra, and the protein can be targeted to the dCas9 variant binding site using the methods and compositions described herein. In some embodiments, the Csy4 is catalytically inactive. In some embodiments, the Cas9 variant, preferably a dCas9 variant, is fused to FokI as described in U.S. Pat. No. 8,993,233; US 20140186958; U.S. Pat. No. 9,023,649; WO/2014/099744; WO 2014/089290; WO2014/144592; WO144288; WO2014/204578; WO2014/152432; WO2115/099850; U.S. Pat. No. 8,697,359; US2010/0076057; US2011/0189776; US2011/0223638; US2013/0130248; WO/2008/108989; WO/2010/054108; WO/2012/164565; WO/2013/098244; WO/2013/176772; US20150050699; US 20150071899 and WO 2014/204578.

Linkers and Tags

In some embodiments, the fusion proteins include a linker between the nuclease and the AP. Linkers that can be used in these fusion proteins (or between fusion proteins in a concatenated structure) can include any sequence that does not interfere with the function of the fusion proteins. In preferred embodiments, the linkers are short, e.g., 2-20 amino acids, and are typically flexible (i.e., comprising amino acids with a high degree of freedom such as glycine, alanine, and serine). In some embodiments, the linker comprises one or more units consisting of GGGS (SEQ ID NO:3) or GGGGS (SEQ ID NO:4), e.g., two, three, four, or more repeats of the GGGS (SEQ ID NO:5) or GGGGS (SEQ ID NO:6) unit. Other linker sequences can also be used, e.g., SSGNSNANSRGPSFSSGLVPLSLRGSH.

In some embodiments, the fusion protein includes a cell-penetrating peptide sequence that facilitates delivery to the intracellular space, e.g., HIV-derived TAT peptide, penetratins, transportans, or hCT derived cell-penetrating peptides, see, e.g., Caron et al., (2001) Mol Ther. 3(3):310-8; Langel, Cell-Penetrating Peptides: Processes and Applications (CRC Press, Boca Raton Fla. 2002); El-Andaloussi et al., (2005) Curr Pharm Des. 11(28):3597-611; and Deshayes et al., (2005) Cell Mol Life Sci. 62(16):1839-49.

Cell penetrating peptides (CPPs) are short peptides that facilitate the movement of a wide range of biomolecules across the cell membrane into the cytoplasm or other organelles, e.g. the mitochondria and the nucleus. Examples of molecules that can be delivered by CPPs include therapeutic drugs, plasmid DNA, oligonucleotides, siRNA, peptide-nucleic acid (PNA), proteins, peptides, nanoparticles, and liposomes. CPPs are generally 30 amino acids or less, are derived from naturally or non-naturally occurring protein or chimeric sequences, and contain either a high relative abundance of positively charged amino acids, e.g. lysine or arginine, or an alternating pattern of polar and non-polar amino acids. CPPs that are commonly used in the art include Tat (Frankel et al., (1988) Cell. 55:1189-1193, Vives et al., (1997) J. Biol. Chem. 272:16010-16017), penetratin (Derossi et al., (1994) J. Biol. Chem. 269:10444-10450), polyarginine peptide sequences (Wender et al., (2000) Proc. Natl. Acad. Sci. USA 97:13003-13008, Futaki et al., (2001) J. Biol. Chem. 276:5836-5840), and transportan (Pooga et al., (1998) Nat. Biotechnol. 16:857-861).

CPPs can be linked with their cargo through covalent or non-covalent strategies. Methods for covalently joining a CPP and its cargo are known in the art, e.g. chemical cross-linking (Stetsenko et al., (2000) J. Org. Chem. 65:4900-4909, Gait et al. (2003) Cell. Mol. Life. Sci. 60:844-853) or cloning a fusion protein (Nagahara et al., (1998) Nat. Med. 4:1449-1453). Non-covalent coupling between the cargo and short amphipathic CPPs comprising polar and non-polar domains is established through electrostatic and hydrophobic interactions.

CPPs have been utilized in the art to deliver potentially therapeutic biomolecules into cells. Examples include cyclosporine linked to polyarginine for immunosuppression (Rothbard et al., (2000) Nature Medicine 6(11):1253-1257), siRNA against cyclin B1 linked to a CPP called MPG for inhibiting tumorigenesis (Crombez et al., (2007) Biochem Soc. Trans. 35:44-46), tumor suppressor p53 peptides linked to CPPs to reduce cancer cell growth (Takenobu et al., (2002) Mol. Cancer Ther. 1(12):1043-1049, Snyder et al., (2004) PLoS Biol. 2:E36), and dominant negative forms of Ras or phosphoinositol 3 kinase (PI3K) fused to Tat to treat asthma (Myou et al., (2003) J. Immunol. 171:4399-4405).

CPPs have been utilized in the art to transport contrast agents into cells for imaging and biosensing applications. For example, green fluorescent protein (GFP) attached to Tat has been used to label cancer cells (Shokolenko et al., (2005) DNA Repair 4(4):511-518). Tat conjugated to quantum dots have been used to successfully cross the blood-brain barrier for visualization of the rat brain (Santra et al., (2005) Chem. Commun. 3144-3146). CPPs have also been combined with magnetic resonance imaging techniques for cell imaging (Liu et al., (2006) Biochem. and Biophys. Res. Comm. 347(1):133-140). See also Ramsey and Flynn, Pharmacol Ther. 2015 Jul. 22. pii: S0163-7258(15)00141-2.

Alternatively, or in addition, the fusion proteins can include a nuclear localization sequence, e.g., SV40 large T antigen NLS (PKKKRRV (SEQ ID NO:7)) and nucleoplasmin NLS (KRPAATKKAGQAKKKK (SEQ ID NO:8)). Other NLSs are known in the art; see, e.g., Cokol et al., EMBO Rep. 2000 Nov. 15; 1(5): 411-415; Freitas and Cunha, Curr Genomics. 2009 December; 10(8): 550-557.

In some embodiments, the fusion proteins include a moiety that has a high affinity for a ligand, for example GST, FLAG or hexahistidine sequences. Such affinity tags can facilitate the purification of recombinant variant proteins.

For methods in which the fusion proteins are delivered to cells, the fusion proteins can be produced using any method known in the art, e.g., by in vitro translation, or expression in a suitable host cell from nucleic acid encoding the variant protein; a number of methods are known in the art for producing proteins. For example, the fusion proteins can be produced in and purified from yeast, E. coli, insect cell lines, plants, transgenic animals, or cultured mammalian cells; see, e.g., Palomares et al., “Production of Recombinant Proteins: Challenges and Solutions,” Methods Mol Biol. 2004; 267:15-52. In addition, the fusion proteins can be linked to a moiety that facilitates transfer into a cell, e.g., a lipid nanoparticle, optionally with a linker that is cleaved once the protein is inside the cell. See, e.g., LaFountaine et al., Int J Pharm. 2015 Aug. 13; 494(1):180-194.

Expression Systems

To use the fusion proteins described herein, it may be desirable to express them from a nucleic acid that encodes them. This can be performed in a variety of ways. For example, a nucleic acid encoding the fusion proteins can be cloned into an intermediate vector for transformation into prokaryotic or eukaryotic cells for replication and/or expression. Intermediate vectors are typically prokaryote vectors, e.g., plasmids, or shuttle vectors, or insect vectors, for storage or manipulation of the nucleic acid encoding the fusion proteins for production of the fusion proteins. The nucleic acid encoding the fusion proteins can also be cloned into an expression vector, for administration to a plant cell, animal cell, preferably a mammalian cell or a human cell, fungal cell, bacterial cell, or protozoan cell.

To obtain expression, a nucleic acid sequence encoding a fusion protein is typically subcloned into an expression vector that contains a promoter to direct transcription. Suitable bacterial and eukaryotic promoters are well known in the art and described, e.g., in Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 2010). Bacterial expression systems for expressing the engineered protein are available in, e.g., E. coli, Bacillus sp., and Salmonella (Palva et al., 1983, Gene 22:229-235). Kits for such expression systems are commercially available. Eukaryotic expression systems for mammalian cells, yeast, and insect cells are well known in the art and are also commercially available.

The promoter used to direct expression of a nucleic acid depends on the particular application. For example, a strong constitutive promoter is typically used for expression and purification of fusion proteins. In contrast, when the fusion protein is to be administered in vivo for gene regulation, either a constitutive or an inducible promoter can be used, depending on the particular use of the fusion protein. In addition, a preferred promoter for administration of the fusion protein can be a weak promoter, such as HSV TK or a promoter having similar activity. The promoter can also include elements that are responsive to transactivation, e.g., hypoxia response elements, Gal4 response elements, lac repressor response element, and small molecule control systems such as tetracycline-regulated systems and the RU-486 system (see, e.g., Gossen & Bujard, 1992, Proc. Natl. Acad. Sci. USA, 89:5547; Oligino et al., 1998, Gene Ther., 5:491-496; Wang et al., 1997, Gene Ther., 4:432-441; Neering et al., 1996, Blood, 88:1147-55; and Rendahl et al., 1998, Nat. Biotechnol., 16:757-761).

In addition to the promoter, the expression vector typically contains a transcription unit or expression cassette that contains all the additional elements required for the expression of the nucleic acid in host cells, either prokaryotic or eukaryotic. A typical expression cassette thus contains a promoter operably linked, e.g., to the nucleic acid sequence encoding the fusion protein, and any signals required, e.g., for efficient polyadenylation of the transcript, transcriptional termination, ribosome binding sites, or translation termination. Additional elements of the cassette may include, e.g., enhancers, and heterologous spliced intronic signals.

The particular expression vector used to transport the genetic information into the cell is selected with regard to the intended use of the fusion protein, e.g., expression in plants, animals, bacteria, fungus, protozoa, etc. Standard bacterial expression vectors include plasmids such as pBR322 based plasmids, pSKF, pET23D, and commercially available tag-fusion expression systems such as GST and LacZ.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG; pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

The vectors for expressing the fusion proteins can include RNA Pol III promoters to drive expression of the guide RNAs, e.g., the H1, U6 or 7SK promoters. These human promoters allow for expression of fusion proteins in mammalian cells following plasmid transfection.

Some expression systems have markers for selection of stably transfected cell lines such as thymidine kinase, hygromycin B phosphotransferase, and dihydrofolate reductase. High yield expression systems are also suitable, such as using a baculovirus vector in insect cells, with the gRNA encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of recombinant sequences. Standard transfection methods are used to produce bacterial, mammalian, yeast or insect cell lines that express large quantities of protein, which are then purified using standard techniques (see, e.g., Colley et al., 1989, J. Biol. Chem., 264:17619-22; Guide to Protein Purification, in Methods in Enzymology, vol. 182 (Deutscher, ed., 1990)). Transformation of eukaryotic and prokaryotic cells are performed according to standard techniques (see, e.g., Morrison, 1977, J. Bacteriol. 132:349-351; Clark-Curtiss & Curtiss, Methods in Enzymology 101:347-362 (Wu et al., eds, 1983).

Any of the known procedures for introducing foreign nucleotide sequences into host cells may be used. These include the use of calcium phosphate transfection, polybrene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the fusion protein.

The present invention also includes nucleic acids, vectors and cells comprising the vectors described herein.

Kits

Also provided herein are kits for use in the methods described herein. The kits can include one or more of the following: a vector encoding a site-specific nuclease with an AP linked in-frame or with one or more cloning sites for inclusion of an AP; purified recombinant nuclease proteins; guide RNAs (e.g., produced in vitro), e.g., as controls, when necessary; reagents for use with the nuclease, optionally including control template DNA and/or guide RNA; and/or instructions for use in a method described herein.

### EXAMPLES

The invention is further described in the following examples, which do not limit the scope of the invention described in the claims.

### Example #1

### Epigenetically Regulated Sequence-Specific Nucleases

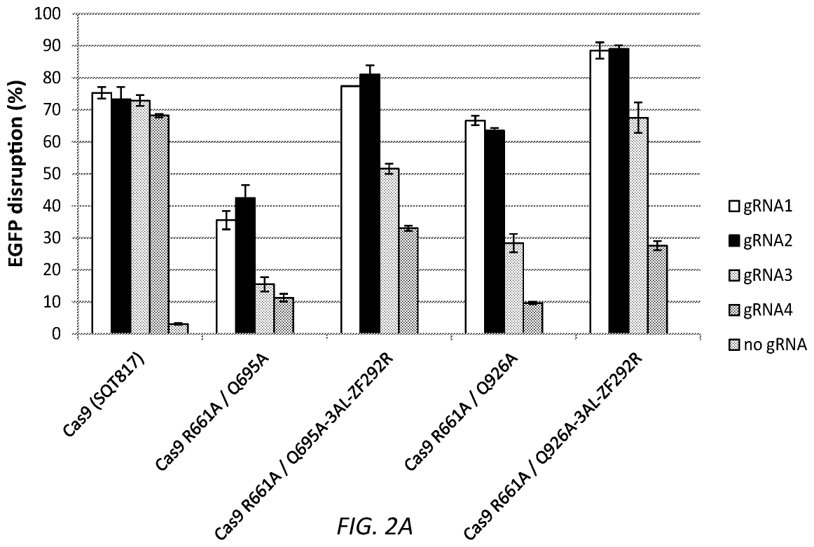

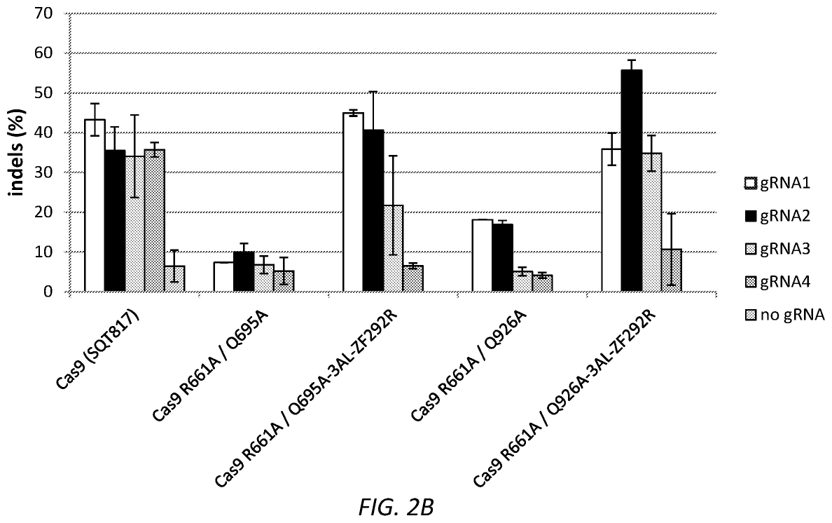

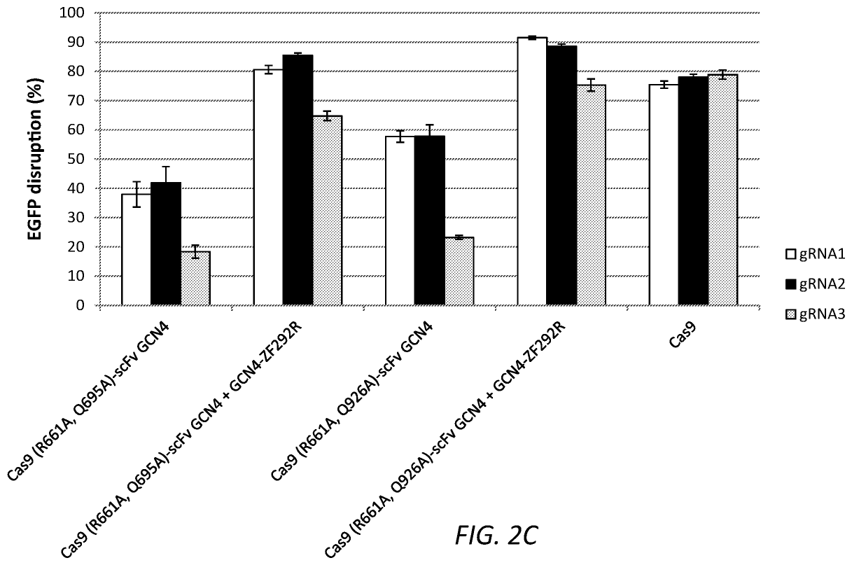

A system was developed in which SpCas9 variants bearing R661A and Q695A mutations or bearing R661A and Q926A mutations were genetically fused to an engineered zinc finger array (ZF292R) targeted to a genomically integrated single copy EGFP reporter gene. Introduction of a nuclease-induced DSB into the EGFP coding region that is then repaired via NHEJ can lead to the introduction of frameshift mutations, causing cells to become EGFP-negative, a phenotype that can be quantitatively assayed using flow cytometry. We tested the activities of these variant nucleases with and without the ZF292R zinc finger array together with four different gRNA variants targeting the same site in EGFP: (1) gRNA with 20 nt of homology to the target site and with an additional 5′ appended G that is mismatched to the target site sequence (gRNA1), (2) gRNA with 19 nt of homology to the target site and a 5′ 20^{th} nt that is a G, which is mismatched to the target site (gRNA2), (3) gRNA with 18 nt of homology to the target site with two 5′ Gs mismatched to the target site (gRNA3), and (4) a perfectly matched gRNA with 17 nt of homology to the target site and no additional mismatched G nts (gRNA4). When tested with all four gRNAs, SpCas9 (R661A, Q695A) and SpCas9 (R661A, Q926A) both showed increased nuclease activity when fused to ZF292R as judged by EGFP disruption assay (FIG. 2A). We also performed TIDE, a sequencing-based indel quantification assay, to directly assess the nuclease activity of each of these nuclease complexes. In agreement with the flow cytometry assay, analysis of the cell populations by TIDE demonstrated increased rates of indel formation when both SpCas9 variants were fused to ZF292R with all four gRNAs tested (FIG. 2B).

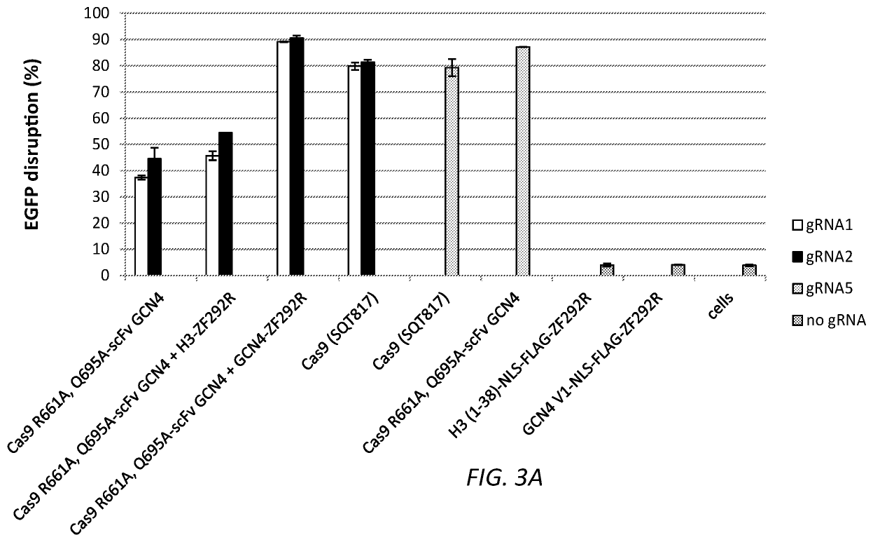

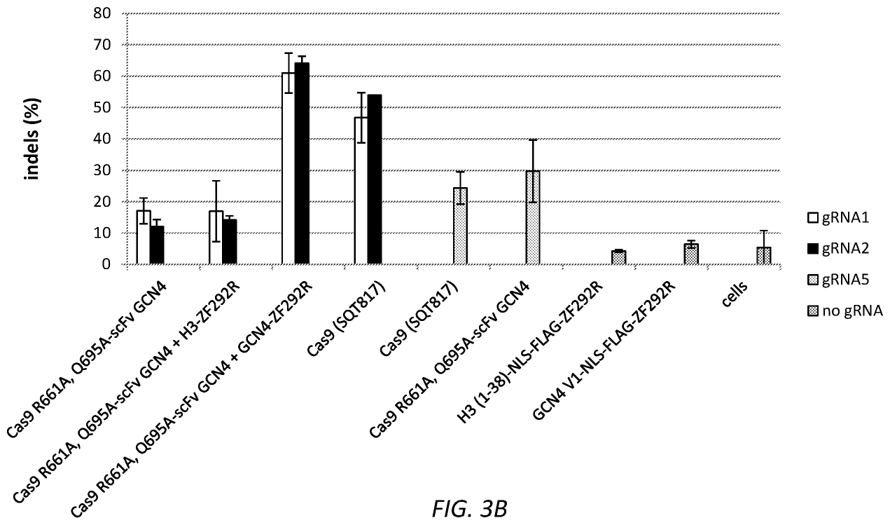

To provide proof of principle for creating nucleases with activities dependent on binding to a DNA-bound artificial transcription factor, we next developed a system in which ZF292R is genetically fused to a GCN4 peptide (GCN4-ZF292R) that can be bound tightly and specifically by an engineered scFv (scFv GCN4). We fused this scFv GCN4 directly to SpCas9 (R661A, Q695A) and SpCas9 (R661A, Q926A) and evaluated whether these SpCas9-scFv GCN4 fusions were able to disrupt EGFP in the presence or absence of the GCN4-ZF292R fusion using gRNA1, gRNA2, or gRNA3 (FIG. 2C). Both SpCas9 (R661A, Q695A)-scFv GCN4 and SpCas9 (R661A, Q926A)-scFv GCN4 showed enhanced EGFP disruption as determined by flow cytometry when co-expressed with GCN4-ZF292R. To determine whether this activity was specific to the interaction between GCN4-ZF292R and scFv GCN4, we performed a second experiment in which SpCas9 (R661A, Q695A)-scFv GCN4 was co-expressed with GCN4-ZF292R or H3 (1-38)-ZF292R (a fusion of the same ZF292R zinc finger array to the N-terminal 38 amino acids of histone H3). Indeed, SpCas9 (R661A, Q695A)-scFv GCN4 demonstrated increased EGFP disruption when co-expressed with GCN4-ZF292R but not with H3 (1-38)-ZF292R using gRNA1 and gRNA 2 (FIG. 3A). In agreement with the flow cytometry assay, analysis of these cell populations by TIDE demonstrated increased rates of indel formation by SpCas9 (R661A, Q695A)-scFv GCN4 only when co-expressed with GCN4-ZF292R and not H3 (1-38)-ZF292R (FIG. 3B). Additionally, as a control, each SpCas9 fusion construct was tested with a gRNA bearing 20 nt of perfect complementarity to a different target site in EGFP with no appended 5′ mismatched G (gRNA5) to ensure that the proteins retained nuclease activity comparable to wild-type SpCas9 in the absence of the above gRNA modifications.

### Example #2

### Sequence-Specific Nucleases That Depend on Three-Dimensional Chromatin Conformation

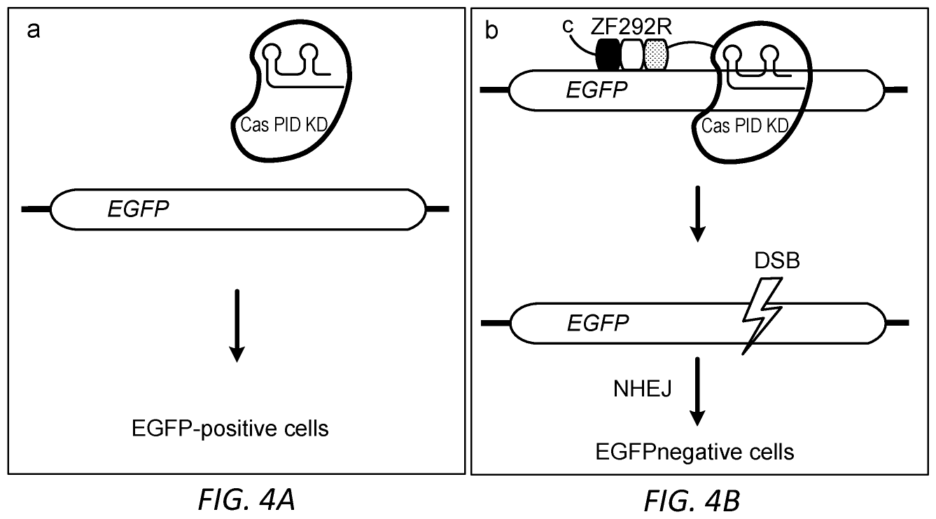

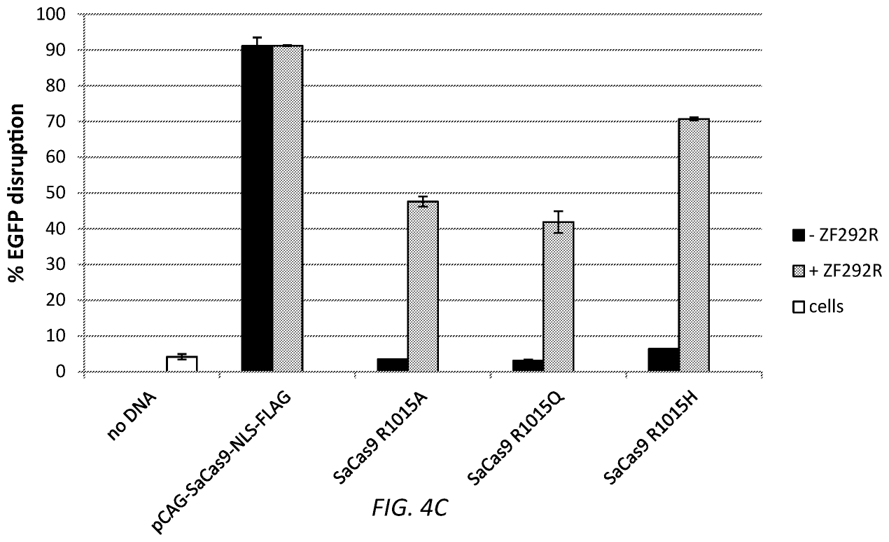

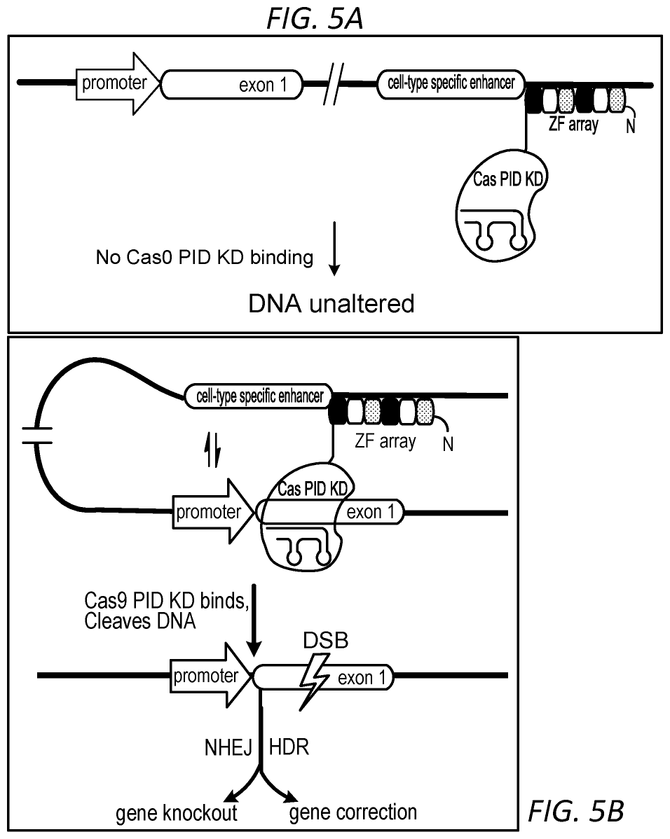

Previous work has shown that SpCas9 can be engineered to induce DSBs only when tethered near its target site by a second DNA binding domain (DBD) such as an engineered zinc finger array (ZF) or TALE repeat array. This is accomplished by introducing mutations into SpCas9 at positions R1333 or R1335 that affect the ability of the protein to recognize its PAM motif (such mutants are termed Cas9 PAM interacting domain knock-downs or Cas9 PID KDs). Using an EGFP disruption assay similar to the one described in Strategy #1, we have shown that an analogous system with SaCas9 can be engineered by fusing a second ZF DBD to a SaCas9 PID KDs bearing the mutations R1015A, R1015Q, or R1015H, which affect the interaction between SaCas9 and the PAM sequence at the target site (FIGS. 4A and 4B). To test this, we tested fusions of SaCas9 variants bearing an R1015A, R1015Q, or R1015H mutation targeted to a site in the EGFP reporter gene that is adjacent to the binding site of the ZF292R domain using a gRNA harboring 21 nts of complementarity to the target site. Fusions of these SaCas9 variants to the ZF292R DBD restored significant EGFP disruption activity to these nucleases (FIG. 4C). For this invention, we envision fusing SpCas9 or SaCas9 PID KDs to an engineered ZF or TALE that binds to a DNA sequence distal to the Cas9 target site in linear sequence but that is only proximal in three-dimensional space in specific cell types. Thus, with this configuration, cell-type-specific chromatin looping between the distal sequence targeted by the second DBD and the target site of the Cas9 PID KD will bring the nuclease in close proximity to the gRNA target site, causing the Cas9 PID KD to induce a DSB at the target gene (FIGS. 5A and 5B). Furthermore, in lieu of Cas9 PID KDs, we propose fusing the SpCas9 variants outlined in Table 1 to an engineered DBD targeted to distal regulatory sequences. Using the gRNA modifications outlined in Strategy #1 and Strategy #2, we would be able to achieve nuclease activity from the SpCas9 variants only when the second DBD is able to bind to its target site proximal to the gRNA target site (e.g., only in those cell types in which there is looping between the distal regulatory element and the gene of interest).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of modifying the genome of a cell, the method comprising expressing in the cell, or contacting the cell with, a fusion protein comprising a targeted nuclease that is linked to an engineered affinity protein (AP) that possesses high affinity for a specific transcription factor (TF) or post-translational histone modification.

2. The method of claim 1, wherein the AP is selected from the group consisting of single chain antibodies, engineered fibronectin domains, engineered Staphylococcus aureus immunoglobulin binding protein A, engineered nanobodies, and designed Ankyrin repeat proteins.

3. The method of claim 1, wherein the nuclease is selected from the group consisting of 1) meganucleases, 2) zinc-finger nucleases, 3) transcription activator effector-like nucleases (TALEN), and 4) Clustered Regularly Interspaced Short Palindromic Repeats (CRISPR)-CRISPR-associated (Cas) or CRISPR-Cpf1 RNA-guided nuclease (RGN).

4. The method of claim 3, wherein when the nuclease is a CRISPR-Cas or CRISPR-Cpf1 RGN and the method is performed in the presence of a guide RNA.

5. The method of claim 4 wherein the nuclease is a Streptococcus pyogenes Cas9 nuclease harboring mutation of one or more of the residues shown in Table 1.

6. A method of modifying the genome of a cell, the method comprising expressing in the cell, or contacting the cell with, a fusion protein comprising a zinc finger DNA binding domain (ZF DBD) or TAL DNA binding array fused to a Staphylococcus aureus Cas9 comprising a mutation at R1015.

7. The method of claim 6, wherein the S. aureus Cas9 comprises a mutation selected from the group consisting of R1015A, R1015Q, and R1015H.

8. A method of modifying the genome of a cell, the method comprising expressing in the cell, or contacting the cell with, a fusion protein comprising (i) a targeted DNA binding domain or a catalytically inactive “dead” RGN (dRGN) with a guide RNA, (ii) a heterologous functional domain, and (iii) an engineered affinity protein (AP) that is only active if a transcription factor or histone modification recognized by the AP is present proximal to the target site of the DNA binding domain or dRGN.

9. The method of claim 8, wherein the AP is selected from the group consisting of single chain antibodies, engineered fibronectin domains, engineered Staphylococcus aureus immunoglobulin binding protein A, engineered nanobodies, and designed Ankyrin repeat proteins.

10. The method of claim 9, wherein the functional domain is a transcriptional regulatory domain, a histone modifying enzyme, or a DNA modifying enzyme.

11. The method of claim 4, wherein the guide RNA is selected from the group consisting of (i) gRNAs with spacer lengths of 19, 18, and 17 bp; (ii) gRNAs possessing one, two, or three intentional mismatches relative to the intended target site; (iii) gRNAs with 20 nts of complementarity to the on-target site, with an additional 5′ G base (that is mismatched to the target DNA sequence) appended; and (iv) a combination of any of (i)-(iii).

12. The method of claim 8, wherein the guide RNA is a truncated gRNA bearing very short complementarity sequences to the target DNA of 9, 10, 11, 12, or 13 nucleotide bases.

13. The method of claim 8, wherein the guide RNA is selected from the group consisting of (i) gRNAs with spacer lengths of 19, 18, and 17 bp; (ii) gRNAs possessing one, two, or three intentional mismatches relative to the intended target site; (iii) gRNAs with 20 nts of complementarity to the on-target site, with an additional 5′ G base (that is mismatched to the target DNA sequence) appended; and (iv) a combination of any of (i)-(iii).
